(19) United States
(12) Patent Application Publication
(10) Pub. No.: US 2008/0038245 A1
(43) Pub. Date: Feb. 14, 2008

(54) Methods of modulating angiogenesis

(76) Inventor: George L. King, Dover, MA (US)

Correspondence Address: FISH & RICHARDSON PC, P.O. BOX 1022, MINNEAPOLIS, MN, 55440-1022, US

(21) Appl. No.: 11/646,933

(22) Filed: Dec. 28, 2006

Related U.S. Application Data
(63) Continuation of application No. 10/107,956, filed on Mar. 26, 2002, now abandoned.
(60) Provisional application No. 60/279,083, filed on Mar. 27, 2001.

(51) Int. Cl. A61K 38/45; A61K 31/407; C12N 5/00; A61K 31/7088

(52) U.S. Cl. 424/94.5; 435/375; 514/410; 514/44

(57) ABSTRACT

Method of modulating angiogenesis in a cell, tissue or subject and methods of treating an angiogenesis-related disorder include modulating PKCβ activity.

### RELATED APPLICATIONS

This application is a continuation of U.S. Ser. No. 10/107,956, filed Mar. 26, 2002, which claims the benefit of U.S. Provisional Application Ser. No. 60/279,083, filed Mar. 27, 2001, the contents of which are incorporated herein by reference in their entirety.

### BACKGROUND

Neovascularization or angiogenesis is a complex process involving numerous growth factors and multiple signaling pathways. Vascular endothelial growth factor (VEGF) has been reported to be the most potent growth factor and plays central roles in development of vascular tissue and angiogenesis in diseases such as tumor, rheumatoid arthritis, and proliferative diabetic retinopathy.

### SUMMARY OF THE INVENTION

Protein kinase C (PKC) is thought to affect VEGF activity, and PKC activation has been reported to be involved in some diabetic vascular complications. Neovascular diseases of the retina are major causes of blindness worldwide. The inventors have discovered that modulating PKC activity, e.g., PKCβ activity, e.g., PKCβ2 activity, can modulate neovascular response, e.g., angiogenesis, in a cell, tissue, or subject, e.g., a retinal cell or tissue (e.g., a schemic retinal tissue), a tumor tissue, an arthritic tissue, or a human or non-human subject, e.g., an experimental rodent, e.g., an animal model of retinopathy.

The inventors have found that PKCβ, e.g., PKCβ2, enhances VEGF-induced cell proliferation, e.g., in retinal cells, e.g., in schemic retinas. Without wanting to be bound by theory, the inventors believe that PKCβ, e.g., PKCβ2, increases phosphorylation of the Rb suppressor in response to VEGF, leading to inactivation of Rb. This leads to increased transcription and increased proliferation of vascular endothelial cells and/or smooth muscle cells (SMC) and increased angiogenesis.

Accordingly, the invention features a method of modulating cell growth, e.g., endothelial cell growth or SMC growth (e.g., angiogenesis) in a cell, tissue, or subject, e.g., a retinal tissue, e.g., an ischemic retina, a tumor tissue, an arthritic tissue, or a human or non-human subject. The method includes modulating PKCβ, e.g., PKCβ2, activity in the cell, tissue, or subject. PKCβ activity can be modulated by, e.g., modulating transcription of the PKCβ gene, modulating PKCβ protein levels, or modulating PKCβ activity.

In a preferred embodiment, PKCβ, e.g., PKCβ2, levels, activity or expression is decreased, thereby decreasing phosphorylation of Rb. Dephosphorylation increases Rb's transcriptional suppressor activity, leading to decreased transcription, decreased proliferation of vascular endothelial cells or SMC and less angiogenesis. PKCβ levels, activity or expression can be decreased by administering an agent that inhibits PKCβ protein, activity or expression levels, e.g., by administering a PKC antagonist (e.g., a PKCβ antagonist). An agent that inhibits PKC protein levels, activity or expression can be one or more of: (a) a polypeptide, e.g., an antibody (e.g., an intrabody) that specifically binds to and inhibits PKC, e.g., PKCβ, or specifically binds to a PKCβ substrate, e.g., Rb (e.g., the polypeptide sterically hinders phosphorylation of Rb by PKCβ at one or more of: S249/T252, S780, S795, and S821); (b) an agent that decreases PKC, e.g., PKCβ, gene expression, e.g., a small molecule which binds the promoter of PKC, e.g., PKCβ; (c) a mutated, inactive PKC that exhibits a dominant negative effect on PKC, e.g., PKCβ, signaling, e.g., a kinase inactive PKCβ; (d) a chemical compound, e.g., an organic compound, e.g., a naturally occurring or synthetic organic compound that decreases PKC, e.g., PKCβ signaling; (e) a nucleic acid molecule that binds to a cellular PKC, e.g., PKCβ, nucleic acid sequence, e.g., mRNA, and inhibits expression of the protein, e.g., a PKCβ antisense molecule, ribozyme, dsRNA, or siRNA. In another preferred embodiment, PKC, e.g., PKCβ, is inhibited by decreasing the level of expression of an endogenous PKC gene (e.g., PKCβ), e.g., by decreasing transcription of the PKC gene (e.g., PKCβ). In a preferred embodiment, transcription of the PKC gene can be decreased by: altering the regulatory sequences of the endogenous PKC gene, e.g., by the addition of a negative regulatory sequence (such as a DNA-binding site for a transcriptional repressor). In another preferred embodiment, the level of expression of an endogenous PKC gene (e.g., a PKCβ gene) is decreased by: an event which disrupts expression of the PKC gene, e.g., such as a knock in or knockout of the PKC gene (e.g., PKCβ gene).

In a preferred embodiment, the PKCβ inhibitory agent is LY333531 (Science 1996 May 3; 272(5262):728-31).

In a preferred embodiment, the cell, tissue, or subject is diseased, e.g., the tissue is a cancer tissue or an ischemic tissue.

In a preferred embodiment, the cell or tissue is retinal tissue, e.g., ischemic retina.

In a preferred embodiment, the subject has or is at risk for an angiogenesis-related disorder, e.g., retinopathy, e.g., oxygen-induced retinopathy-of-prematurity, oxygen-induced retinopathy, diabetic retinopathy, retinopathy associated with retinal vein occlusion, or sickle cell retinopathy. The subject can be a human or non-human animal, e.g., an animal model of retinopathy of prematurity, e.g., as described in Penn et al. (2001) Invest Ophthalmol Vis Sci 42:283-90.

In a preferred embodiment, PKCβ is inhibited in-vitro, e.g., in an isolated cell or tissue of a subject, e.g., an isolated retinal cell or tissue. In some embodiments, the cell or tissue can be transplanted into a subject. The transplanted cell or tissue can be autologous, allogeneic, or xenogeneic.

In another preferred embodiment, PKCβ signaling is decreased in-vivo in a subject.

In a preferred embodiment, the agent is targeted to a retinal tissue in a subject.

In preferred embodiments, the method includes identifying a subject as being in need of treatment or prevention of an angiogenesis-related disorder, e.g., retinopathy (e.g., oxygen-induced retinopathy-of-prematurity, oxygen-induced retinopathy, or diabetic retinopathy), a tumor, or arthritis.

In some embodiments, a second therapeutic agent is administered to the subject, e.g., an antibiotic agent, an anti-diabetic agent, or another inhibitor of PKCβ, e.g., a second agent described herein above.

In a preferred embodiment, the administration of the agent can be initiated, e.g., (a) when the subject begins to show signs of an angiogenesis-related disorder, e.g., retinopathy, e.g., a retinopathy described herein; (b) when an angiogenesis-related disorder, e.g., retinopathy, e.g., a retinopathy described herein, is diagnosed; (c) before, during or after a treatment for an angiogenesis-related disorder, e.g., retinopathy, e.g., a retinopathy described herein, is begun or begins to exert its effects; or (d) generally, as is needed to maintain health, e.g., normal vision. The period over which the agent is administered (or the period over which clinically effective levels are maintained in the subject) can be long term, e.g., for six months or more or a year or more, or short term, e.g., for less than a year, six months, one month, two weeks or less.

In another embodiment, PKCβ, e.g., PKCβ2, levels, activity and/or expression are increased, thereby increasing phosphorylation of Rb. Phosphorylation decreases Rb's transcriptional suppressor activity, leading to increased transcription and increased proliferation of vascular endothelial cells or SMC and increased angiogenesis. PKCβ, e.g., PKCβ2, levels, activity or expression can be increased by administering an agent that promotes, increases or mimics the expression, level or activity of PKCβ, e.g., PKCβ2. The agent can be one or more of: (a) a PKCβ polypeptide or a functional fragment or analog thereof, e.g., a PKCβ2 polypeptide or a functional fragment or analog thereof; (b) an agent that increases PKC, e.g., PKCβ, nucleic acid expression, e.g., a small molecule which binds to the promoter region of PKC, e.g., PKCβ; (c) a peptide or protein agonist of PKC, e.g., PKCβ, that increases PKC kinase activity; (d) an antibody, e.g., an antibody that binds to and stabilizes or assists the binding of PKCβ, e.g., PKCβ2, to a binding partner; (e) a chemical compound, e.g., an organic compound, e.g., a naturally occurring or synthetic organic compound that increases expression of PKCβ, e.g., PKCβ2; or (f) a nucleotide sequence encoding a PKCβ, e.g., PKCβ2, polypeptide or functional fragment or analog thereof. The nucleotide sequence can be a genomic sequence or a cDNA sequence. The nucleotide sequence can include: a PKCβ, e.g., PKCβ2, coding region; a promoter sequence, e.g., a promoter sequence from a PKCβ, e.g., PKCβ2, gene or from another gene; an enhancer sequence; untranslated regulatory sequences, e.g., a 5′ untranslated region (UTR), e.g., a 5′UTR from a PKCβ, e.g., PKCβ2, gene or from another gene, a 3′ UTR, e.g., a 3′UTR from a PKCβ, e.g., PKCβ2, gene or from another gene; a polyadenylation site; an insulator sequence. In another preferred embodiment, the level of a PKCβ, e.g., PKCβ2, is increased by increasing the level of expression of an endogenous a PKCβ, e.g., PKCβ2 gene, e.g., by increasing transcription of the a PKCβ, e.g., PKCβ2, gene or increasing PKCβ, e.g., PKCβ2, mRNA stability. In a preferred embodiment, transcription of the PKCβ, e.g., PKCβ2, gene is increased by: altering the regulatory sequence of the endogenous a PKCβ gene, e.g., in an retinal cell, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the a PKCβ, e.g., PKCβ2, gene to be transcribed more efficiently.

In a preferred embodiment, PKCβ is increased in-vitro, e.g., in an isolated cell or tissue of a subject, e.g., an isolated retinal cell or tissue. In some embodiments, the cell or tissue can be transplanted into a subject. The transplanted cell or tissue can be autologous, allogeneic, or xenogeneic.

In another preferred embodiment, PKCβ signaling is increased in-vivo in a subject.

In a preferred embodiment, the agent is targeted to a retinal tissue in a subject.

In preferred embodiments, the method includes identifying a subject as being in need of treatment or prevention of an angiogenesis-related disorder, e.g., a disorder characterized by insufficient vascularization. The subject can be a human or non-human animal, e.g., an animal model of retinopathy of prematurity, e.g., as described in Penn et al. (2001) Invest Ophthalmol Vis Sci 42:283-90.

In some embodiments, a second therapeutic agent is administered to the subject, e.g., an antibiotic agent, an anti-diabetic agent, or another promoer of PKCβ, e.g., a second agent described herein above.

In a preferred embodiment, the administration of the agent can be initiated, e.g., (a) when the subject begins to show signs of an angiogenesis-related disorder; (b) when an angiogenesis-related disorder is diagnosed; (c) before, during or after a treatment for an angiogenesis-related disorder is begun or begins to exert its effects; or (d) generally, as is needed to maintain health. The period over which the agent is administered (or the period over which clinically effective levels are maintained in the subject) can be long term, e.g., for six months or more or a year or more, or short term, e.g., for less than a year, six months, one month, two weeks or less.

In another aspect, the invention features a method of modulating cell growth such as endothelial cell growth or SMC growth, e.g., angiogenesis, in a cell, tissue, or subject, e.g., a retinal tissue, e.g., an ischemic retina, a tumor tissue, an arthritic tissue, or a human or non-human subject. The method includes modulating an Rb activity in the cell, tissue, or subject. An Rb activity can be any of: transcriptional repressor activity, tumor suppressor activity, anti-proliferation activity, interaction (e.g., binding) with E2F transcription factor, or inactivation of E2F activity.

In a preferred embodiment, an Rb activity is increased, thereby decreasing cell growth, e.g., endothelial cell growth or SMC growth, e.g., angiogenesis. Rb activity can be increased by, e.g., administering an agent that increases, promotes or mimics Rb activity. An agent that increases, promotes or mimics Rb activity can be one or more of: (a) an agent that decreases phosphorylation of Rb, e.g., a phosphatase, or an inhibitor of a kinase that acts on Rb, e.g., an inhibitor of PKCβ, e.g., PKCβ2, e.g., an inhibitor of PKCβ described herein; (b) an agent that increases, promotes or stabilizes an interaction, e.g., binding, between Rb and E2F, or between Rb and PKCβ, e.g., an antibody that stabilizes Rb-E2F binding; (c) an Rb polypeptide or a functional fragment or analog thereof; (d) an agent that increases Rb nucleic acid expression, e.g., a small molecule which binds to the promoter region of Rb; (e) a peptide or protein agonist of Rb that increases an Rb activity; (f) an antibody, e.g., an antibody that binds to and stabilizes or assists the binding of Rb to a binding partner, e.g., PKCβ or E2F; (g) a chemical compound, e.g., an organic compound, e.g., a naturally occurring or synthetic organic compound that increases expression of Rb; or (h) a nucleotide sequence encoding an Rb polypeptide or functional fragment or analog thereof. The nucleotide sequence can be a genomic sequence or a cDNA sequence. The nucleotide sequence can include: an Rb coding region; a promoter sequence, e.g., a promoter sequence from an Rb gene or from another gene; an enhancer sequence; untranslated regulatory sequences, e.g., a 5′ untranslated region (UTR), e.g., a 5′UTR from an Rb gene or from another gene, a 3′ UTR, e.g., a 3′UTR from an Rb gene or from another gene; a polyadenylation site; an insulator sequence. In another preferred embodiment, the level of Rb is increased by increasing the level of expression of an endogenous Rb gene, e.g., by increasing transcription of the Rb gene or increasing Rb mRNA stability. In a preferred embodiment, transcription of an Rb gene is increased by: altering the regulatory sequence of the endogenous Rb gene, e.g., in an retinal cell, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the Rb gene to be transcribed more efficiently.

In another embodiment, an Rb activity is decreased, thereby increasing cell growth, e.g., endothelial cell growth or SMC growth, e.g., angiogenesis. Rb activity can be decreased by, e.g., administering an agent that decreases or inhibits Rb activity. An agent that decreases or inhibits Rb activity can be one or more of: (a) an agent that increases Rb phosphorylation, e.g., a kinase, e.g., PKCβ, or agent that increases a kinase activity, e.g., an agent described herein that increases, promotes or mimics PKCβ, e.g., PKCβ2, activity, levels or expression (e.g., an agent that increases phosphorylation of Rb at one or more of: S249/T242, S780, S795, and S821); (b) an agent, e.g., a polypeptide, e.g., an antibody, that inhibits the interaction, e.g., binding, between Rb and E2F or between Rb and PKCβ; (c) a mutated, inactive Rb that exhibits a dominant negative effect on an Rb activity, e.g., an Rb that binds PKCβ but does not bind E2F; (d) a chemical compound, e.g., an organic compound, e.g., a naturally occurring or synthetic organic compound that decreases Rb signaling; (e) a nucleic acid molecule that binds to a cellular Rb nucleic acid sequence, e.g., mRNA, and inhibits expression of the protein, e.g., an Rb antisense molecule, ribozyme, dsRNA, or siRNA. In another preferred embodiment, Rb is inhibited by decreasing the level of expression of an endogenous Rb gene, e.g., by decreasing transcription of the Rb gene. In a preferred embodiment, transcription of the Rb gene can be decreased by: altering the regulatory sequences of the endogenous Rb gene, e.g., by the addition of a negative regulatory sequence (such as a DNA-binding site for a transcriptional repressor). In another preferred embodiment, the level of expression of an endogenous Rb gene is decreased by: an event which disrupts expression of the Rb gene, e.g., such as a knock in or knockout of the Rb gene.

In a preferred embodiment, the agent that increases Rb activity is a specific inhibitor of PKCβ, e.g., LY-333531 (Science 1996 May 3; 272(5262):728-31).

In a preferred embodiment, the cell, tissue, or subject is diseased, e.g., the tissue is a cancer tissue or an ischemic tissue.

In a preferred embodiment, the cell or tissue is retinal tissue, e.g., ischemic retina.

In a preferred embodiment, the subject has or is at risk for an angiogenesis-related disorder, e.g., retinopathy, e.g., oxygen-induced retinopathy-of-prematurity, oxygen-induced retinopathy, or diabetic retinopathy. The subject can be a human or non-human animal, e.g., an animal model of retinopathy of prematurity, e.g., as described in Penn et al. (2001) Invest Ophthalmol Vis Sci 42:283-90.

In a preferred embodiment, Rb activity is increased in-vitro, e.g., in an isolated cell or tissue of a subject, e.g., an isolated retinal cell or tissue. In some embodiments, the cell or tissue can be transplanted into a subject. The transplanted cell or tissue can be autologous, allogeneic, or xenogeneic.

In another preferred embodiment, Rb activity is increased in-vivo in a subject.

In a preferred embodiment, the agent is targeted to a retinal tissue in a subject.

In preferred embodiments, the method includes identifying a subject as being in need of treatment or prevention of an angiogenesis-related disorder, e.g., retinopathy, e.g., oxygen-induced retinopathy-of-prematurity, oxygen-induced retinopathy, or diabetic retinopathy.

In some embodiments, a second therapeutic agent is administered to the subject, e.g., an antibiotic agent, an anti-diabetic agent, or another agent that increases Rb activity, e.g., another inhibitor of PKCβ.

In a preferred embodiment, the administration of the agent can be initiated, e.g., (a) when the subject begins to show signs of an angiogenesis-related disorder, e.g., retinopathy, e.g., a retinopathy described herein; (b) when an angiogenesis-related disorder, e.g., retinopathy, e.g., a retinopathy described herein, is diagnosed; (c) before, during or after a treatment for an angiogenesis-related disorder, e.g., retinopathy, e.g., a retinopathy described herein, is begun or begins to exert its effects; or (d) generally, as is needed to maintain health, e.g., normal vision. The period over which the agent is administered (or the period over which clinically effective levels are maintained in the subject) can be long term, e.g., for six months or more or a year or more, or short term, e.g., for less than a year, six months, one month, two weeks or less.

In another aspect, the invention features a method of treating a disorder, e.g., a tumor, rheumatoid arthritis, or a diabetes related disorder, e.g., diabetes mellitus, diabetic retinopathy, hyperglycemia, or diabetic nephropathy in a subject. The method includes modulating PKCβ activity in a cell or tissue of the subject. In a preferred embodiment, the method includes administering an agent that inhibits PKCβ, e.g., PKCβ2, activity, levels or expression, thereby resulting in decreased angiogenesis in e.g., a tumor or a retina. PKCβ can be decreased by any of the agents described herein for decreasing PKCβ activity or expression.

In a preferred embodiment, the subject is a human.

In a preferred embodiment, the subject is a non-human animal, e.g., an animal model of retinopathy of prematurity, e.g., as described in Penn et al. (2001) Invest Ophthalmol Vis Sci 42:283-90.

In a preferred embodiment, the disorder is diabetes mellitus.

In a preferred embodiment, the disorder is retinopathy.

In a preferred embodiment, the disorder is cancer or a tumor.

In a preferred embodiment, the agent is LY-333531.

In a preferred embodiment, the method includes identifying the subject as having or being at risk for an angiogenesis-related disorder, e.g., retinopathy, e.g., oxygen-induced retinopathy-of-prematurity, oxygen-induced retinopathy, or diabetic retinopathy.

In a preferred embodiment, PKCβ activity is decreased in-vitro, e.g., in an isolated cell or tissue of a subject, e.g., an isolated retinal cell or tissue. In some embodiments, the cell or tissue can be transplanted into a subject. The transplanted cell or tissue can be autologous, allogeneic, or xenogeneic.

In another preferred embodiment, PKCβ activity is decreased in-vivo in a subject.

In a preferred embodiment, the agent is targeted to a retinal tissue in the subject.

In some embodiments, a second therapeutic agent is administered to the subject, e.g., an antibiotic agent, an anti-diabetic agent, or another agent that decreases PKCβ activity, e.g., another inhibitor of PKCβ.

In a preferred embodiment, the administration of the agent can be initiated, e.g., (a) when the subject begins to show signs of an angiogenesis-related disorder, e.g., retinopathy, e.g., a retinopathy described herein; (b) when an angiogenesis-related disorder, e.g., retinopathy, e.g., a retinopathy described herein, is diagnosed; (c) before, during or after a treatment for an angiogenesis-related disorder, e.g., retinopathy, e.g., a retinopathy described herein, is begun or begins to exert its effects; or (d) generally, as is needed to maintain health, e.g., normal vision. The period over which the agent is administered (or the period over which clinically effective levels are maintained in the subject) can be long term, e.g., for six months or more or a year or more, or short term, e.g., for less than a year, six months, one month, two weeks or less.

In another aspect, the invention features a method of treating a disorder, e.g., a tumor, rheumatoid arthritis, or a diabetes related disorder, e.g., diabetes mellitus, diabetic retinopathy, hyperglycemia, or diabetic nephropathy. The method includes modulating an Rb activity in a cell or tissue of the subject. In a preferred embodiment, the method includes administering an agent that increases, promotes or mimics Rb activity, thereby resulting in decreased angiogenesis in e.g., a tumor or a retina. Rb activity can be increased by any of the agents described herein for increasing, promoting or mimicking Rb activity, levels or expression.

In a preferred embodiment, the agent decreases Rb phosphorylation.

In another preferred embodiment, the agent increases, promotes or mimics the interaction, e.g., binding, between Rb and E2F. Preferably, an agent which decreases Rb phosphorylation is also an agent which decreases PKCβ activity or expression.

In a preferred embodiment, the subject is a human.

In a preferred embodiment, the subject is a non-human animal, e.g., an animal model of retinopathy of prematurity, e.g., as described in Penn et al. (2001) Invest Ophthalmol Vis Sci 42:283-90.

In a preferred embodiment, the disorder is diabetes mellitus.

In a preferred embodiment, the disorder is retinopathy.

In a preferred embodiment, the disorder is cancer or a tumor.

In a preferred embodiment, the agent is a PKCβ inhibitor, e.g., LY-333531.

In a preferred embodiment, the method includes identifying the subject as having or being at risk for an angiogenesis-related disorder, e.g., retinopathy, e.g., oxygen-induced retinopathy-of-prematurity, oxygen-induced retinopathy, or diabetic retinopathy.

In a preferred embodiment, Rb activity is increased in-vitro, e.g., in an isolated cell or tissue of a subject, e.g., an isolated retinal cell or tissue. In some embodiments, the cell or tissue can be transplanted into a subject. The transplanted cell or tissue can be autologous, allogeneic, or xenogeneic.

In another preferred embodiment, Rb activity is increased in-vivo in a subject.

In a preferred embodiment, the agent is targeted to a retinal tissue in the subject.

In some embodiments, a second therapeutic agent is administered to the subject, e.g., an antibiotic agent, an anti-diabetic agent, or another agent that decreases PKCβ activity, e.g., another inhibitor of PKCβ.

In a preferred embodiment, the administration of the agent can be initiated, e.g., (a) when the subject begins to show signs of an angiogenesis-related disorder, e.g., retinopathy, e.g., a retinopathy described herein; (b) when an angiogenesis-related disorder, e.g., retinopathy, e.g., a retinopathy described herein, is diagnosed; (c) before, during or after a treatment for an angiogenesis-related disorder, e.g., retinopathy, e.g., a retinopathy described herein, is begun or begins to exert its effects; or (d) generally, as is needed to maintain health, e.g., normal vision. The period over which the agent is administered (or the period over which clinically effective levels are maintained in the subject) can be long term, e.g., for six months or more or a year or more, or short term, e.g., for less than a year, six months, one month, two weeks or less.

In a preferred embodiment, a pharmaceutical composition including an agent described herein is administered in a therapeutically effective dose. The invention also features the use of an agent or pharmaceutical composition described herein in the manufacture of a medicament for the treatment or prevention of cancer, rheumatoid arthritis, or a diabetes related disorder, e.g., diabetes mellitus, diabetic retinopathy, hyperglycemia, or diabetic nephropathy, or an angiogenesis related disorder, e.g., an angiogenesis-related disorder described herein.

In another aspect, the invention features a method of evaluating a subject, e.g., determining if a subject is at risk for, or has, an angiogenesis related disorder, e.g., cancer, retinopathy, e.g., diabetic retinopathy, proliferative diabetic retinopathy, retinopathy of prematurity, retinopathy associated with retinal vein occlusion, sickle cell retinopathy, or radiation-induced disorder. The method includes evaluating PKCβ and/or Rb activity, levels or expression in a cell or tissue, preferably in the eye, of the subject. Abnormal or aberrant PKCβ and/or Rb activity, levels or expression in the subject as compared to a control can indicate the risk or presence of an angiogenesis related disorder (e.g., an angiogenesis related disorder described herein) in the subject. For example, increased PKCβ activity, levels or expression and/or decreased Rb activity, levels or expression, compared to a control, can indicate the risk or presence of an ocular disorder, e.g., an ocular disorder described herein.

In a preferred embodiment, the method includes detecting a genetic lesion or mutation in a PKCβ and/or Rb gene. The PKCβ and Rb, e.g., human PKCβ and Rb gene sequences are known in the art.

In a preferred embodiment, the method includes evaluating the level of expression of a PKCβ and/or Rb gene, e.g., evaluating the amount or half life of a PKCβ and/or Rb mRNA, e.g., Over- or under-expression of a PKCβ and/or Rb gene, compared to a control, can be evaluated by, e.g., Northern blot, TaqMan assay, or other methods known in the art.

In a preferred embodiment, the method includes evaluating a PKCβ and/or Rb activity, e.g., PKC kinase activity, or Rb-E2F binding activity.

In a preferred embodiment, the method includes evaluating protein levels of a PKCβ and/or Rb protein.

In a preferred embodiment, the method includes treating the subject for the angiogenesis related disorder.

In a preferred embodiment, the subject is further evaluated for one or more of the following parameters: (1) vision; (2) glucose levels; (3) insulin level.

In a preferred embodiment, the evaluation is used to choose a course of treatment.

Methods of the invention can be used prenatally or to determine if a subject's offspring will be at risk for a disorder.

In another aspect, the invention features a method of evaluating an agent, e.g., screening for an agent that modulates angiogenesis, e.g., in the eye. The method includes (a) providing a test agent, and (b) determining if the agent interacts with a PKCβ and/or Rb, e.g., binds to and/or modulates the levels, expression, or activity of PKCβ and/or Rb e.g., determining if it modulates the ability of PKCβ and/or Rb to interact with a ligand. Agents, e.g., compounds, identified by this method can be used, e.g., in the treatment of an angiogenesis related disorder, e.g., cancer, a retinopathy, e.g., diabetic retinopathy, proliferative diabetic retinopathy, retinopathy of prematurity, retinopathy associated with retinal vein occlusion, sickle cell retinopathy.

In one embodiment, the method includes: providing a PKCβ and/or Rb protein or nucleic acid, or a functional fragment thereof; contacting the PKCβ and/or Rb protein or nucleic acid with a test agent, and determining if the test compound interacts with, e.g., binds, the PKCβ and/or Rb protein or nucleic acid.

In one embodiment, the test agent binds to the PKCβ and/or Rb protein and modulates a PKCβ and/or Rb activity, e.g., a PKCβ and/or Rb activity described herein. For example, the compound binds to the PKCβ and/or Rb protein and facilitates or inhibits any of: kinase activity or binding activity, e.g., Rb-E2F binding activity. Methods for assaying PKCβ activity or binding activity, e.g., methods described herein, are art-recognized.

In a preferred embodiment, the test compound is one or more of: a protein or peptide; an antibody; a small molecule; a nucleotide sequence. For example, the agent can be an agent identified through a library screen described herein.

In a preferred embodiment, the contacting step is performed in vitro.

In another preferred embodiment, the contacting step is performed in vivo.

In a preferred embodiment, the method further includes administering the test compound to an experimental animal, e.g., an animal model for an angiogenesis related disorder, e.g., a cancer, or an angiogenesis related disorder described herein, e.g., a retinopathy described herein. In a preferred embodiment, the animal model is an animal model of retinopathy of prematurity, e.g., as described in Penn et al. (2001) Invest Ophthalmol Vis Sci 42:283-90.

In another embodiment, the method includes: providing a test cell, tissue, or subject; administering a test agent to the cell, tissue, or subject; and determining whether the test agent modulates a PKCβ and/or Rb expression, level or activity in the cell, tissue, or subject. An agent that is found to modulate a PKCβ and/or Rb activity in the cell, tissue, or subject is identified as an agent that can modulate angiogenesis or vascularization, e.g., neovascularization, in the subject, e.g., in the eye.

In a preferred embodiment, the cell is a retinal cell.

In a preferred embodiment, the method includes (a) providing a cell-free expression system, cell, tissue, or animal having a transgene which includes a nucleic acid that encodes a reporter molecule functionally linked to the control region, e.g., a promoter, of a gene encoding a PKCβ and/or Rb; (b) contacting the cell-free expression system, cell, tissue, or animal with a test agent; and (c) evaluating a signal produced by the reporter molecule. A test agent that causes the modulation of reporter molecule expression, compared to a reference, e.g., a negative control, is identified as an agent that can modulate angiogenesis, e.g., in the eye. Preferred agents decrease expression of a PKCβ and/or increase expression of Rb, where the reporter molecule is under the control of a control region from a gene encoding PKCβ and/or Rb.

In a preferred embodiment, the reporter molecule is any of: green fluorescent protein (GFP); enhanced GFP (EGFP); luciferase; chloramphenicol acetyl transferase (CAT); β-galactosidase; β-lactamase; or secreted placental alkaline phosphatase. Other reporter molecules, e.g., other enzymes whose function can be detected by appropriate chromogenic or fluorogenic substrates are known to those skilled in the art.

In a preferred embodiment, the agent is further tested in a cell-based and/or animal based model e.g., a cell based or animal model described herein for an angiogenesis related disorder.

In another aspect, the invention features a method of evaluating a subject, e.g., determining if a subject (e.g., a human) is at risk for or has an angiogenesis related disorder, e.g., a retinopathy, e.g., a retinopathy described herein. The method includes evaluating PKCβ, Rb or E2F in the subject. An abnormality, e.g., a lower or higher than normal expression, level or activity of PKCβ, Rb or E2F, being indicative of risk.

In one embodiment, the method includes: (a) evaluating (i) the level of PKCβ, Rb or E2F and/or (ii) an activity of PKCβ, Rb or E2F; and optionally (b) comparing the level and/or activity to a reference, e.g., a control, e.g., the level and/or activity in a tissue from a subject known not to have a retinopathy. An activity of PKCβ, Rb or E2F can include a PKCβ, Rb or E2F activity described herein, e.g., a binding activity, kinase activity, transcriptional repressor activity or transcriptional activation activity. The method can also include evaluating the subject for a symptom of an angiogenesis related disorder, e.g., a retinopathy.

In a preferred embodiment, the subject is a human.

In a preferred embodiment, the method includes treating the subject for the disorder.

In a preferred embodiment, the evaluation is used to choose a course of treatment.

In another aspect, the invention features a computer readable record encoded with (a) a subject identifier, e.g., a patient identifier, (b) one or more results from an evaluation of the subject, e.g., a diagnostic evaluation described herein, e.g., the level of expression, level or activity of PKCβ and/or Rb, in the subject, and optionally (c) a value for or related to a disease state, e.g., a value correlated with disease status or risk with regard to an ocular disorder, e.g., an ocular disorder described herein. In one embodiment, the invention features a computer medium having a plurality of digitally encoded data records. Each data record includes a value representing the level of expression, level or activity of PKCβ and/or Rb, in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression, level or activity of genes other than PKCβ and/or Rb (e.g., other genes associated with an angiogenesis related disorder, or other genes on an array). The data record can be structured as a table, e.g., a table that is part of a database such as a relational database (e.g., a SQL database of the Oracle or Sybase database environments). The invention also includes a method of communicating information about a subject, e.g., by transmitting information, e.g., transmitting a computer readable record described herein, e.g., over a computer network.

In another aspect, the invention features a method of providing information, e.g., for making a decision with regard to the treatment of a subject having, or at risk for, an angiogenesis related disorder described herein. The method includes (a) evaluating the expression, level or activity of PKCβ and/or Rb; optionally (b) providing a value for the expression, level or activity of PKCβ and/or Rb; optionally (c) comparing the provided value with a reference value, e.g., a control or non-disease state reference or a disease state reference; and optionally (d) based, e.g., on the relationship of the provided value to the reference value, supplying information, e.g., information for making a decision on or related to the treatment of the subject.

In a preferred embodiment, the provided value relates to an activity described herein, e.g., to a kinase activity of PKCβ, or a binding activity, e.g., a Rb-E2F binding activity.

In a preferred embodiment, the decision is whether to administer a preselected treatment.

In a preferred embodiment, the decision is whether a party, e.g., an insurance company, HMO, or other entity, will pay for all or part of a preselected treatment.

Also featured is a method of evaluating a sample. The method includes providing a sample, e.g., from the subject, and determining a gene expression profile of the sample, wherein the profile includes a value representing the level of expression of PKCβ and/or Rb. The method can further include comparing the value or the profile (i.e., multiple values) to a reference value or reference profile. The gene expression profile of the sample can be obtained by methods known in the art (e.g., by providing a nucleic acid from the sample and contacting the nucleic acid to an array). The method can be used to diagnose an angiogenesis related disorder, e.g., a disorder described herein, in a subject wherein misexpression of PKCβ and/or Rb, e.g., an increase in PKCβ expression or a decrease in Rb expression, is an indication that the subject has or is disposed to having an angiogenesis related disorder. The method can be used to monitor a treatment for an angiogenesis related disorder in a subject. For example, the gene expression profile can be determined for a sample from a subject undergoing treatment. The profile can be compared to a reference profile or to a profile obtained from the subject prior to treatment or prior to onset of the disorder (see, e.g., Golub et al. (1999) Science 286:531).

In another aspect, the invention features a method of evaluating a gene for its involvement in an angiogenesis related disorder, e.g., cancer or a retinopathy described herein. The method includes (a) providing a cell, tissue, or animal in which PKCβ and/or Rb mediated signaling, e.g., PKCβ and/or Rb -mediated angiogenesis signaling, is perturbed, e.g., PKCβ and/or Rb described herein is perturbed, (b) evaluating the expression of one or more genes in the cell, tissue, or animal, and (c) optionally comparing the expression of the one or more genes in the cell, tissue, or animal with a reference, e.g., with the expression of the one or more genes in a control cell, tissue or animal. A gene or genes identified as increased or decreased in the cell, tissue, or animal as compared to the reference, e.g., the control, are identified as candidate genes involved in an ocular disorder, e.g., an ocular disorder described herein.

In a preferred embodiment, the cell or tissue is from a subject (e.g., a human or non-human animal, e.g., an experimental animal) having or being at risk for an ocular disorder, e.g., an ocular disorder described herein.

In a preferred embodiment, the animal is a transgenic animal, e.g., a transgenic animal having a knock-out or overexpressing mutation for PKCβ and/or Rb.

In yet another aspect, the invention features a method of evaluating a test compound. The method includes providing a cell and a test compound; contacting the test compound to the cell; obtaining a subject expression profile for the contacted cell; and comparing the subject expression profile to one or more reference profiles. The profiles include a value representing the level of expression of PKCβ and/or Rb. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an uncontacted cell.

In another aspect, the invention features, a method of evaluating a subject. The method includes: a) obtaining a sample from a subject, e.g., from a caregiver, e.g., a caregiver who obtains the sample from the subject; b) determining a subject expression profile for the sample. Optionally, the method further includes either or both of steps: c) comparing the subject expression profile to one or more reference expression profiles; and d) selecting the reference profile most similar to the subject reference profile. The subject expression profile and the reference profiles include a value representing the level of expression of PKCβ and/or Rb. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of expression of PKCβ and/or Rb.

As used herein, “treatment” or “treating a subject” is defined as the application or administration of a therapeutic agent to a patient, or application or administration of a therapeutic agent to an isolated tissue or cell line from a patient, e.g., a retinal cell or tissue, who has a disease, a symptom of disease or a predisposition toward a disease, e.g., an angiogenesis related disorder, e.g., cancer or retinopathy, e.g., a retinopathy described herein. Treatment can slow, cure, heal, alleviate, relieve, alter, remedy, ameliorate, improve or affect the disease, a symptom of the disease or the predisposition toward disease, e.g., by at least 10%.

As used herein, to ability of a first molecule to “interact” with a second molecule refers to the ability of the first molecule to act upon the structure and/or activity of the second molecule, either directly or indirectly. For example, a first molecule can interact with a second by (a) directly binding, e.g., specifically binding, the second molecule, e.g., transiently or stably binding the second molecule; (b) modifying the second molecule, e.g., by cleaving a bond, e.g., a covalent bond, in the second molecule, or adding or removing a chemical group to or from the second molecule, e.g., adding or removing a phosphate group or carbohydrate group; (c) modulating an enzyme that modifies the second molecule, e.g., inhibiting or activating a kinase or phosphatase that normally modifies the second molecule; (d) affecting expression of the second molecule, e.g., by binding, activating, or inhibiting a control region of a gene encoding the second molecule, or binding, activating, or inhibiting a transcription factor that associates with the gene encoding the second molecule; (d) affecting the stability of an mRNA encoding the second molecule, e.g., by inhibiting mRNAse activity against the mRNA encoding the second molecule or by degrading the mRNA encoding the second molecule.

### DETAILED DESCRIPTION

Retinal neovascularization is a major cause of blindness and requires the activities of several signaling pathways and multiple cytokines. Activation of protein kinase C (PKC) enhances the angiogenic process and is involved in the signaling of vascular endothelial growth factor (VEGF). The data described herein demonstrates a dramatic increase in the angiogenic response to oxygen-induced retinal ischemia in transgenic mice overexpressing PKCβ2 isoform and a significant decrease in retinal neovascularization in PKCβ isoform null mice. The mitogenic action of VEGF, a potent hypoxia-induced angiogenic factor, was increased by 2-fold in retinal endothelial cells by the overexpression of PKCβ1 or β2 isoforms and inhibited significantly by the overexpression of a dominant-negative PKCβ2 isoform but not by the expression of PKC α, δ, and ζ isoforms. Association of PKCβ2 isoform with retinoblastoma protein was discovered in retinal endothelial cells, and PKCβ2 isoform increased retinoblastoma phosphorylation under basal and VEGF-stimulated conditions. The potential functional consequences of PKCβ-induced retinoblastoma phosphorylation could include enhanced E2 promoter binding factor transcriptional activity and increased VEGF-induced endothelial cell proliferation.

Protein Kinase C

Protein kinase C (PKC) is a membrane-associated enzyme that is regulated by a number of factors, including membrane phospholipids, calcium, and membrane lipids such as diacylglycerols that are liberated in response to the activities of phospholipases (Bell et al. J. Biol. Chem. 1991. 266:4661-4664; Nishizuka, Science 1992. 258:607-614. The protein kinase C isozymes, alpha, beta (β)-1, beta-2 and gamma, require membrane phospholipid, calcium and diacylglycerol/phorbol esters for full activation. The delta, epsilon, eta, and theta forms of PKC are calcium-independent in their mode of activation. The zeta and lambda forms of PKC are independent of both calcium and diacylglycerol and are believed to require only membrane phospholipid for their activation. PKC- and isozyme-specific (e.g., PKC β specific) modulators are described, e.g., in Goekjian et al. Current Medicinal Chemistry, 1999, 6:877-903; Way et al., Trends Pharmacol Sci, 2000, 21:181-7, and in U.S. Pat. No. 5,843,935.

Role of PKCβ in Ischemia-Induced Retinal Neovascularization.

The effects of PKCβ isoforms on ischemia-induced retinal neovas-cularization were studied in the oxygen-induced retinopathy-of-prematurity model by using PKCβ-null mice (12) (PKCβ KO mice) or transgenic mice overexpressing PKCβ2 isoform (PKCβTg) in vascular tissues under preproendothelin promoter (9, 10), which exhibited a 9-fold increase in PKC activity. Five days after oxygen treatment, both wild-type and PKCβTg mice developed retinal neovascularization as observed by fluorescein-perfused retinal flat mount. However, a more extensive network of neovascularization was observed in PKCβ Tg mice than with the wild type (B6/FVB). In contrast, much less neovascularization was observed in the retina of PKCβ KO mice than with their respective control mice (B6/129). No retinal neovascularization or significant morphological differences were observed in the vasculature of any groups not receiving oxygen treatment. No differences were observed in the extent of retinal capillary occlusion in mice with oxygen treatment at postnatal day 12. Analysis of retinal cross-sections demonstrated that the number of endothelial cell nuclei anterior to the internal limiting membrane (a measure of the extent of neovascularization) decreased by 55% in PKCβ KO mice and increased by 3.2-fold in PKCβ Tg mice as compared with their respective controls. These data suggested that activation of PKCβ2 isoform contributed significantly to the development of neovascularization in response to hypoxia. However, PKCβ2 isoform is not essential for the angiogenic process during embryonic development or growth, because both of these processes are normal in PKCβ Tg and KO mice. VEGF has been reported to be one of the main growth factors inducing neovascularization in the oxygen-induced retinopathy model (13). Thus, mRNA levels of VEGF and Flk1, a tyrosine kinase receptor primarily mediating VEGF's mitogenic actions in endothelial cells, were measured by Northern blot analysis. No differences were detected in the mRNA levels of VEGF or Flk1 in either PKCβ transgenic mice or knockout mice as compared with their individual controls on postnatal day 14. These data suggest that the angiogenic effects of PKCβ isoform are potentially caused by enhancement of VEGF's intracellular action rather than by increasing the concentration of VEGF or Flk1.

Role of PKC Isoforms in VEGF-Induced Angiogenic Responses.

The effects of various PKC isoforms on VEGF-induced cellular proliferation and migration were correlated with several intra-cellular signaling pathways in BREC. VEGF (0.6 nM) induced a 2-fold increase in BREC total DNA content of BREC after 4 days. The addition of GFX (a general PKC inhibitor) and PD98059 (a MEK inhibitor) blocked VEGF's mitogenic activity completely (2). The effects of each PKC isoform were determined by infecting BREC with nonreplicating adenoviral vectors containing either the wild-type or kinase-inactive dominant-negative mutants of PKC α, β1 and 2, δ, and theta isoforms. (PKCβ1 and β2 had similar effects in all studies with BREC.) Only PKCβ isoforms enhanced VEGF-induced growth in BREC, resulting in an 86% increase. Overexpression of dominant-negative of PKCβ2 isoform inhibited PKCβ activity by 90% and decreased VEGF-induced growth by 68%, suggesting that VEGF's mitogenic activity in BREC depended in part on PKCβ isoform and mitogen-activated protein kinase pathway activation. PKC activation also enhanced VEGF-induced BREC migration. VEGF (0.6 nM) increased migration of BREC 2-fold, an effect inhibited by GFX, PD98059, and (41%) by wortmannin (a phosphatidylinositol 3-kinase (PI3-kinase) inhibitor). Overexpression of PKC α, β2, δ, and theta isoforms by adenoviral vectors increased protein levels of these PKC isoforms by more than 10-fold; however, only PKCβ2 and δ isoforms enhanced VEGF-induced migration, by 100% and 145%, respectively. Overexpression of dominant-negative PKCβ2 and δ isoforms, as described above, inhibited VEGF-induced BREC migration by 55% and 32%, respectively. In addition, overexpression of PKCβ2 and PKCδ isoforms increased VEGF-induced mitogen-activated protein kinase (extracellular signal-regulated kinase; ERK1/2) phosphorylation by 61% and 74%, respectively, but did not increase VEGF-induced PI3-kinase-Akt activation. These data indicated that both PKCβ and PKCδ isoforms mediated VEGF-induced ERK1/2 activation and migration, but only PKCβ isoform enhanced VEGF-induced endothelial cell proliferation, suggesting that these PKC isoforms have both common and distinct targets for downstream actions.

Role of PKCβ in VEGF-Induced Rb-E2F Pathway Activation.

Because PKC activation, particularly the β isoforms, enhanced VEGF's mitogenic activity in BREC, we characterized the effect of overexpressing PKCβ2 isoform on various signaling molecules that regulate the progression of the cell cycle. VEGF and overexpression of PKCβ2, but not PKCδ isoforms increased basal phosphorylation of Rb, a tumor suppressor that can regulate cellular proliferation, differentiation, and death by binding and inactivating the E2F transcriptional factor family (27). VEGF increased phosphorylation of Rb in a time-dependent manner by up to 3-fold at all phosphorylation sites as quantified by various phospho-specific antibodies (S249/T242, S780, S795, S807/S811, and S821). Overexpression of PKCβ2 isoform by adenoviral vectors in-creased basal- and VEGF-induced Rb phosphorylation at S780 and S795 by more than 3-fold, but did not change significantly the phosphorylation at S249/T252, S807/S811, or S821 and did not alter the expression of cyclins or cyclin-dependent kinase inhibitors. Overexpression of PKCδ isoform did not significantly alter VEGF-induced phosphorylation of Rb.

To determine whether PKCβ2 isoform can associate and phosphorylate Rb directly in BREC, immunoprecipitation studies were performed with anti-PKCβ2 antibody, and Rb protein was detected by immunoblot analysis. At basal conditions, some association of PKCβ2 isoform and Rb protein was observed. The addition of VEGF and serum increased the association of PKCβ2 with Rb by 2.2- and 2.6-fold, respectively. Overexpression of PKCβ2 dominant-negative mutant did not associate with Rb. The same complex of PKCβ2 and Rb were characterized with antibodies to pS780 of Rb, which showed a basal phosphorylation with the overexpression of PKCβ2. The addition of VEGF (0.6 nM) increased phosphorylated Rb levels by 2- to 3-fold. Total cell lysates were immunoprecipitated with antibodies to Rb protein and immunoblotted with antibodies to PKCβ2. At basal conditions some associations of PKCβ2 and Rb were observed, but the addition of VEGF (0.6 nM) increased the amount of PKCβ2-associated Rb protein by 1.7-fold. In vitro studies demonstrated that recombinant PKCβ2 isoform phosphorylated Rb protein in the presence of diacylglycerol and phosphatidylserine at S249/T242, S780, S795, and S821 sites. These data suggests that PKCβ2 isoform, when activated, can phosphorylate Rb protein at multiple, but specific sites in an isoform-selective manner.

To determine whether PKCβ2 isoform could increase E2F activity by the phosphorylation of Rb protein, the effect of overexpressing PKCβ2 isoform on VEGF-induced E2F activation was evaluated by luciferase assay in BREC. VEGF and overexpression of PKCβ2 independently increased E2F activity by 8.6- and 3.7-fold, respectively. Overexpression of PKCβ2 increased VEGF's effect by 49%. Again, overexpression of PKCδ isoform was not effective, whereas overexpression of PKCβ2 dominant-negative inhibited VEGF's effect. Finally, inhibition of E2F by E2F double-structured DNA decoy decreased VEGF-induced BREC proliferation in a dose-dependent manner, with a maximum inhibition of 76%.

The data presented herein show that that activation of the β isoforms of PKC can selectively enhance VEGF's mitogenic effects on endothelial cells and hypoxia-induced retinal neovascularization. VEGF's effect on endothelial cell growth differed from its effect on migration, which involved PKCδ isoform and PI3-kinase pathways. The mechanism of the mitogenic activity of PKCβ isoforms on BREC includes the activation of ERK and phosphorylation of Rb protein. These data have identified Rb protein as an isoform-selective target of the activated PKCβ2 isoform in the endothelial cells. The isoform selectivity of PKCβ isoforms could be caused by their subcellular localization that can translocate to the nuclear membrane when activated (28).

VEGF clearly can phosphorylate multiple serine and threonine sites on the Rb protein which are potential phosphorylation sites of cyclin D and E kinases in retinal capillary endothelial cells (29, 30). Phosphorylation of Ser-780, Ser-795, Ser-807, Ser-811, and Thr-821 decreases the binding of Rb to E2F, which would permit endothelial cells to progress from G1 to later stages of the cell cycle (29-32). Activation of PKCβ2 isoform also phosphorylated these serine and threonine sites, except Ser-807 and Ser-811 on the Rb in vitro. However, overexpression of PKCβ2 isoform, but not the PKCδ or the PKCβ2 dominant-negative isoforms, increased only the phosphorylation of Ser-780 and Ser-795 at the C-terminal of Rb but did not phosphorylated other sites induced by PKCβ isoform in vitro or by VEGF. Brown et al. (31) has reported that the loss of Ser-781 and the Ser-788 sites did not affect the binding of Rb to E2F-DNA. However, the phosphorylation of these sites combined with other phosphorylation sites in the N and C terminals affected by VEGF can alter the binding and the promoter activities of E2F on transcription (31, 32). The results of the coprecipitation studies suggested that the activated PKCβ2 but not its kinase-inactive dominant-negative isoform can directly bind and phosphorylate Rb. It is also possible that PKCβ2 isoform could indirectly phosphorylated Ser-780 and Ser-785 of Rb by binding to cyclin E kinase, which has been reported to bind and phosphorylate the same serine sites of Rb (29).

The results described herein have established that the activation of PKCβ isoforms can selectively enhance the mitogenic action of VEGF and hypoxia-induced retinal neovascularization potentially by directly binding to and phosphorylating Rb and subsequently permitting activation of E2F to mediate transcription and cell cycle progression in cells, e.g., in retinal endothelial cells.

Antisense Nucleic Acid Sequences

Nucleic acid molecules which are antisense to a nucleotide encoding PKC, e.g., PKCβ, or Rb, can be used as an agent which inhibits PKC or Rb expression. An “antisense” nucleic acid includes a nucleotide sequence which is complementary to a “sense” nucleic acid encoding the protein to be inhibited e.g., complementary to the coding strand of a double-stranded cDNA molecule or complementary to an mRNA sequence. Accordingly, an antisense nucleic acid can form hydrogen bonds with a sense nucleic acid. The antisense nucleic acid can be complementary to an entire coding strand, or to only a portion thereof. For example, an antisense nucleic acid molecule which antisense to the “coding region” of the coding strand of a nucleotide sequence encoding the protein to be inhibited can be used.

The coding strand sequences encoding PKCβ and Rb are known. Given the coding strand sequences encoding the proteins of interest, antisense nucleic acids can be designed according to the rules of Watson and Crick base pairing. The antisense nucleic acid molecule can be complementary to the entire coding region of mRNA, but more preferably is an oligonucleotide which is antisense to only a portion of the coding or noncoding region of mRNA. For example, the antisense oligonucleotide can be complementary to the region surrounding the translation start site of an mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest.

Administration

An agent, e.g., an Rb agonist or antagonist or a PKC agonist or antagonist, e.g., a PKCβ agonist or antagonist, which modulates the level of expression of an Rb or PKCβ protein can be administered to a subject by standard methods. For example, the agent can be administered by any of a number of different routes including intravenous, intradermal, subcutaneous, oral (e.g., inhalation), transdermal (topical), and transmucosal. In one embodiment, the agent (e.g., a PKC agonist or antagonist) can be administered topically. In a preferred embodiment, the agent is administered to the eye, e.g., as aqueous eye drops or in a cream, lotion or other vehicle suitable for administration onto the eye surface.

The agent which modulates Rb or PKCβ activity, e.g., nucleic acid molecules, Rb or PKCβ polypeptides, fragments or analogs, Rb or PKCβ modulators, and anti-Rb or anti-PKCβ antibodies (also referred to herein as “active compounds”) can be incorporated into pharmaceutical compositions suitable for administration to a subject, e.g., a human. Such compositions typically include the nucleic acid molecule, polypeptide, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for

RNAi

Double stranded nucleic acid molecules that can silence a gene encoding an Rb or PKC, e.g., PKCβ, can also be used as an agent which inhibits expression of PKC signaling in angiogeneis. RNA interference (RNAi) is a mechanism of post-transcriptional gene silencing in which double-stranded RNA (dsRNA) corresponding to a gene (or coding region) of interest is introduced into a cell or an organism, resulting in degradation of the corresponding mRNA. The RNAi effect persists for multiple cell divisions before gene expression is regained. RNAi is therefore an extremely powerful method for making targeted knockouts or “knockdowns” at the RNA level. RNAi has proven successful in human cells, including human embryonic kidney and HeLa cells (see, e.g., Elbashir et al. Nature 2001 May 24; 411(6836):494-8). In one embodiment, gene silencing can be induced in mammalian cells by enforcing endogenous expression of RNA hairpins (see Paddison et al., 2002, PNAS USA 99:1443-1448). In another embodiment, transfection of small (21-23 nt) dsRNA specifically inhibits gene expression (reviewed in Caplen (2002) Trends in Biotechnology 20:49-51).

Briefly, RNAi is thought to work as follows. dsRNA corresponding to a portion of a gene to be silenced is introduced into a cell. The dsRNA is digested into 21-23 nucleotide siRNAs, or short interfering RNAs. The siRNA duplexes bind to a nuclease complex to form what is known as the RNA-induced silencing complex, or RISC. The RISC targets the homologous transcript by base pairing interactions between one of the siRNA strands and the endogenous mRNA. It then cleaves the mRNA ˜12 nucleotides from the 3′ terminus of the siRNA (reviewed in Sharp et al (2001) Genes Dev 15: 485-490; and Hammond et al. (2001) Nature Rev Gen 2: 110-119).

RNAi technology in gene silencing utilizes standard molecular biology methods. dsRNA corresponding to the sequence from a target gene to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Gene silencing effects similar to those of RNAi have been reported in mammalian cells with transfection of a mRNA-cDNA hybrid construct (Lin et al., Biochem Biophys Res Commun 2001 Mar. 2; 281(3):639-44), providing yet another strategy for gene silencing.

Peptide Mimetics

The invention also provides for production of the protein binding domains of PKC, e.g., PKCβ, or Rb, to generate mimetics, e.g. peptide or non-peptide agents, e.g., inhibitory agents. See, for example, “Peptide inhibitors of human papillomavirus protein binding to retinoblastoma gene protein” European patent applications EP 0 412 762 and EP 0 031 080.

Non-hydrolyzable peptide analogs of critical residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), b-turn dipeptide cores (Nagai et al. (1985) Tetrahedron Lett 26:647; and Sato et al. (1986) J Chem Soc Perkin Trans 1:1231), and b-aminoalcohols (Gordon et al. (1985) Biochem Biophys Res Commun 126:419; and Dann et al. (1986) Biochem Biophys Res Commun 134:71).

Antibodies

An agent described herein, e.g., an agent that inhibits or promotes PKC, e.g., PKCβ, or Rb, can also be an antibody specifically reactive with PKC, e.g., PKCβ, or Rb. An antibody can be an antibody or a fragment thereof, e.g., an antigen binding portion thereof. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

The term “antigen-binding fragment” of an antibody (or simply “antibody portion,” or “fragment”), as used herein, refers to one or more fragments of a full-length antibody that retain the ability to specifically bind to an antigen (e.g., a polypeptide encoded by a nucleic acid of Group I or II). Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate nucleic acids, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. The term “monoclonal antibody” or “monoclonal antibody composition”, as used herein, refers to a population of antibody molecules that contain only one species of an antigen binding site capable of immunoreacting with a particular epitope. A monoclonal antibody composition thus typically displays a single binding affinity for a particular protein with which it immunoreacts.

Anti-protein/anti-peptide antisera or monoclonal antibodies can be made as described herein by using standard protocols (See, for example, Antibodies: A Laboratory Manual ed. by Harlow and Lane (Cold Spring Harbor Press: 1988)).

PKC, e.g., PKCβ, or Rb, or a portion or fragment thereof, can be used as an immunogen to generate antibodies that bind the component using standard techniques for polyclonal and monoclonal antibody preparation. The full-length component protein can be used or, alternatively, antigenic peptide fragments of the component can be used as immunogens.

Typically, a peptide is used to prepare antibodies by immunizing a suitable subject, (e.g., rabbit, goat, mouse or other mammal) with the immunogen. An appropriate immunogenic preparation can contain, for example, a recombinant PKC, e.g., PKCβ, or Rb, or a chemically synthesized PKC, e.g., PKCβ, or Rb peptide or anagonist. See, e.g., U.S. Pat. No. 5,460,959; and co-pending U.S. applications U.S. Ser. No. 08/334,797; U.S. Ser. No. 08/231,439; U.S. Ser. No. 08/334,455; and U.S. Ser. No. 08/928,881, which are hereby expressly incorporated by, reference in their entirety. The nucleotide and amino acid sequences of PKC, e.g., PKCβ, and Rb, are known. The preparation can further include an adjuvant, such as Freund's complete or incomplete adjuvant, or similar immunostimulatory agent. Immunization of a suitable subject with an immunogenic component or fragment preparation induces a polyclonal antibody response.

Additionally, antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al., Science 240:1041-1043, 1988; Liu et al., PNAS 84:3439-3443, 1987; Liu et al., J. Immunol. 139:3521-3526, 1987; Sun et al. PNAS 84:214-218, 1987; Nishimura et al., Canc. Res. 47:999-1005, 1987; Wood et al., Nature 314:446-449, 1985; and Shaw et al., J. Natl. Cancer Inst. 80:1553-1559, 1988); Morrison, S. L., Science 229:1202-1207, 1985; Oi et al., BioTechniques 4:214, 1986; Winter U.S. Pat. No. 5,225,539; Jones et al., Nature 321:552-525, 1986; Verhoeyan et al., Science 239:1534, 1988; and Beidler et al., J. Immunol. 141:4053-4060, 1988.

In addition, a human monoclonal antibody directed against PKC, e.g., PKCβ, or Rb, can be made using standard techniques. For example, human monoclonal antibodies can be generated in transgenic mice or in immune deficient mice engrafted with antibody-producing human cells. Methods of generating such mice are describe, for example, in Wood et al. PCT publication WO 91/00906, Kucherlapati et al. PCT publication WO 91/10741; Lonberg et al. PCT publication WO 92/03918; Kay et al. PCT publication WO 92/03917; Kay et al. PCT publication WO 93/12227; Kay et al. PCT publication 94/25585; Rajewsky et al. Pct publication WO 94/04667; Ditullio et al. PCT publication WO 95/17085; Lonberg, N. et al. (1994) Nature 368:856-859; Green, L. L. et al. (1994) Nature Genet. 7:13-21; Morrison, S. L. et al. (1994) Proc. Natl. Acad. Sci. USA 81:6851-6855; Bruggeman et al. (1993) Year Immunol 7:33-40; Choi et al. (1993) Nature Genet. 4:117-123; Tuaillon et al. (1993) PNAS 90:3720-3724; Bruggeman et al. (1991) Eur J Immunol 21:1323-1326); Duchosal et al. PCT publication WO 93/05796; U.S. Pat. No. 5,411,749; McCune et al. (1988) Science 241:1632-1639), Kamel-Reid et al. (1988) Science 242:1706; Spanopoulou (1994) Genes & Development 8:1030-1042; Shinkai et al. (1992) Cell 68:855-868). A human antibody-transgenic mouse or an immune deficient mouse engrafted with human antibody-producing cells or tissue can be immunized with PKC, e.g., PKCβ, or Rb, or an antigenic peptide thereof, and splenocytes from these immunized mice can then be used to create hybridomas. Methods of hybridoma production are well known.

Human monoclonal antibodies can also be prepared by constructing a combinatorial immunoglobulin library, such as a Fab phage display library or a scFv phage display library, using immunoglobulin light chain and heavy chain cDNAs prepared from mRNA derived from lymphocytes of a subject. See, e.g., McCafferty et al. PCT publication WO 92/01047; Marks et al. (1991) J. Mol. Biol. 222:581-597; and Griffths et al. (1993) EMBO J 12:725-734. In addition, a combinatorial library of antibody variable regions can be generated by mutating a known human antibody. For example, a variable region of a human antibody known to bind a PKC, e.g., PKCβ, or Rb, can be mutated, by for example using randomly altered mutagenized oligonucleotides, to generate a library of mutated variable regions which can then be screened to bind to PKC, e.g., PKCβ, or Rb. Methods of inducing random mutagenesis within the CDR regions of immunoglobin heavy and/or light chains, methods of crossing randomized heavy and light chains to form pairings and screening methods can be found in, for example, Barbas et al. PCT publication WO 96/07754; Barbas et al. (1992) Proc. Nat'l Acad. Sci. USA 89:4457-4461.

The immunoglobulin library can be expressed by a population of display packages, preferably derived from filamentous phage, to form an antibody display library. Examples of methods and reagents particularly amenable for use in generating antibody display library can be found in, for example, Ladner et al. U.S. Pat. No. 5,223,409; Kang et al. PCT publication WO 92/18619; Dower et al. PCT publication WO 91/17271; Winter et al. PCT publication WO 92/20791; Markland et al. PCT publication WO 92/15679; Breitling et al. PCT publication WO 93/01288; McCafferty et al. PCT publication WO 92/01047; Garrard et al. PCT publication WO 92/09690; Ladner et al. PCT publication WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) supra; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982. Once displayed on the surface of a display package (e.g., filamentous phage), the antibody library is screened to identify and isolate packages that express an antibody that binds PKC, e.g., PKCβ, or Rb. In a preferred embodiment, the primary screening of the library involves panning with an immobilized PKCβ, Rb or E2F described herein and display packages expressing antibodies that bind immobilized proteins described herein are selected.

Generation of Variants: Production of Altered DNA and Peptide Sequences by Random Methods

Methods are provided herein below for the production of variants of PKC, e.g., PKCβ, or Rb, and for the screening of such variants for a desired activity. Amino acid sequence variants of PKC, e.g., PKCβ, or Rb, or fragments thereof, can be prepared by random mutagenesis of DNA which encodes PKC, e.g., PKCβ, or Rb. Useful methods include PCR mutagenesis and saturation mutagenesis. A library of random amino acid sequence variants can also be generated by the synthesis of a set of degenerate oligonucleotide sequences. One of ordinary skill in the art can use these methods to produce and screen a library, e.g., a library described herein, for the ability to inhibit or promote PKC, e.g., PKCβ, or Rb activity. Assays that can be used to determine if a particular variant has the ability to inhibit or promote PKCβ or Rb are also provided herein below.

PCR Mutagenesis

In PCR mutagenesis, reduced Taq polymerase fidelity is used to introduce random mutations into a cloned fragment of DNA (Leung et al., 1989, Technique 1:11-15). This is a very powerful and relatively rapid method of introducing random mutations. The DNA region to be mutagenized is amplified using the polymerase chain reaction (PCR) under conditions that reduce the fidelity of DNA synthesis by Taq DNA polymerase, e.g., by using a dGTP/dATP ratio of five and adding Mn⁺² to the PCR reaction. The pool of amplified DNA fragments are inserted into appropriate cloning vectors to provide random mutant libraries.

Saturation Mutagenesis

Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as those that alter function, are obtained. The distribution of point mutations is not biased toward conserved sequence elements.

Degenerate Oligonucleotides

A library of homologs can also be generated from a set of degenerate oligonucleotide sequences. Chemical synthesis of a degenerate sequences can be carried out in an automatic DNA synthesizer, and the synthetic genes then ligated into an appropriate expression vector. The synthesis of degenerate oligonucleotides is known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Generation of Variants: Production of Altered DNA and Peptide Sequences by Directed Mutagenesis

Non-random or directed mutagenesis techniques can be used to provide specific sequences or mutations in specific regions. These techniques can be used to create variants that include, e.g., deletions, insertions, or substitutions, of residues of the known amino acid sequence of a protein. The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of options 1-3.

Alanine Scanning Mutagenesis

Alanine scanning mutagenesis is a useful method for identification of certain residues or regions of the desired protein that are preferred locations or domains for mutagenesis, Cunningham and Wells (Science 244:1081-1085, 1989). In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions are then refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

Oligonucleotide-Mediated Mutagenesis

Oligonucleotide-mediated mutagenesis is a useful method for preparing substitution, deletion, and insertion variants of DNA, see, e.g., Adelman et al., (DNA 2:183, 1983). Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. (1978) USA, 75: 5765).

Cassette Mutagenesis

Another method for preparing variants, cassette mutagenesis, is based on the technique described by Wells et al. (Gene, 34:315[1985]). The starting material is a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

Combinatorial Mutagenesis

Combinatorial mutagenesis can also be used to generate mutants. For example, the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

Primary High-Through-Put Methods for Screening Libraries of Peptide Fragments or Homologs

Various techniques are known in the art for screening peptides, e.g., synthetic peptides, e.g., small molecular weight peptides (e.g., linear or cyclic peptides) or generated mutant gene products. Techniques for screening large gene libraries often include cloning the gene library into replicable expression vectors, transforming appropriate cells with the resulting library of vectors, and expressing the genes under conditions in which detection of a desired activity, assembly into a trimeric molecules, binding to natural ligands, e.g., a receptor or substrates, facilitates relatively easy isolation of the vector encoding the gene whose product was detected. Each of the techniques described below is amenable to high through-put analysis for screening large numbers of sequences created, e.g., by random mutagenesis techniques.

Two Hybrid Systems

Two hybrid (interaction trap) assays can be used to identify a protein that interacts with PKC, e.g., PKCβ, or Rb or active fragments thereof These may include, e.g., agonists, superagonists, and antagonists of PKC, e.g., PKCβ, or Rb. (The subject protein and a protein it interacts with are used as the bait protein and fish proteins.). These assays rely on detecting the reconstitution of a functional transcriptional activator mediated by protein-protein interactions with a bait protein. In particular, these assays make use of chimeric genes which express hybrid proteins. The first hybrid comprises a DNA-binding domain fused to the bait protein, e.g., PKC, e.g., PKCβ, or Rb or active fragments thereof. The second hybrid protein contains a transcriptional activation domain fused to a “fish” protein, e.g. an expression library. If the fish and bait proteins are able to interact, they bring into close proximity the DNA-binding and transcriptional activator domains. This proximity is sufficient to cause transcription of a reporter gene which is operably linked to a transcriptional regulatory site which is recognized by the DNA binding domain, and expression of the marker gene can be detected and used to score for the interaction of the bait protein with another protein.

Display Libraries

In one approach to screening assays, the candidate peptides are displayed on the surface of a cell or viral particle, and the ability of particular cells or viral particles to bind an appropriate receptor protein via the displayed product is detected in a “panning assay”. For example, the gene library can be cloned into the gene for a surface membrane protein of a bacterial cell, and the resulting fusion protein detected by panning (Ladner et al., WO 88/06630; Fuchs et al. (1991) Bio/Technology 9:1370-1371; and Goward et al. (1992) TIBS 18:136-140). This technique was used in Sahu et al. (1996) J. Immunology 157:884-891, to isolate a complement inhibitor. In a similar fashion, a detectably labeled ligand can be used to score for potentially functional peptide homologs. Fluorescently labeled ligands, e.g., receptors, can be used to detect homolog which retain ligand-binding activity. The use of fluorescently labeled ligands, allows cells to be visually inspected and separated under a fluorescence microscope, or, where the morphology of the cell permits, to be separated by a fluorescence-activated cell sorter.

A gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at concentrations well over 10¹³ phage per milliliter, a large number of phage can be screened at one time. Second, since each infectious phage displays a gene product on its surface, if a particular phage is recovered from an affinity matrix in low yield, the phage can be amplified by another round of infection. The group of almost identical E. coli filamentous phages M13, fd., and f1 are most often used in phage display libraries. Either of the phage gIII or gVIII coat proteins can be used to generate fusion proteins without disrupting the ultimate packaging of the viral particle. Foreign epitopes can be expressed at the NH₂-terminal end of pIII and phage bearing such epitopes recovered from a large excess of phage lacking this epitope (Ladner et al. PCT publication WO 90/02909; Garrard et al., PCT publication WO 92/09690; Marks et al. (1992) J. Biol. Chem. 267:16007-16010; Griffiths et al. (1993) EMBO J 12:725-734; Clackson et al. (1991) Nature 352:624-628; and Barbas et al. (1992) PNAS 89:4457-4461).

A common approach uses the maltose receptor of E. coli (the outer membrane protein, LamB) as a peptide fusion partner (Charbit et al. (1986) EMBO 5, 3029-3037). Oligonucleotides have been inserted into plasmids encoding the LamB gene to produce peptides fused into one of the extracellular loops of the protein. These peptides are available for binding to ligands, e.g., to antibodies, and can elicit an immune response when the cells are administered to animals. Other cell surface proteins, e.g., OmpA (Schorr et al. (1991) Vaccines 91, pp. 387-392), PhoE (Agterberg, et al. (1990) Gene 88, 37-45), and PAL (Fuchs et al. (1991) Bio/Tech 9, 1369-1372), as well as large bacterial surface structures have served as vehicles for peptide display. Peptides can be fused to pilin, a protein which polymerizes to form the pilus—conduit for interbacterial exchange of genetic information (Thiry et al. (1989) Appl. Environ. Microbiol. 55, 984-993). Because of its role in interacting with other cells, the pilus provides a useful support for the presentation of peptides to the extracellular environment. Another large surface structure used for peptide display is the bacterial motive organ, the flagellum. Fusion of peptides to the subunit protein flagellin offers a dense array of may peptides copies on the host cells (Kuwajima et al. (1988) Bio/Tech. 6, 1080-1083). Surface proteins of other bacterial species have also served as peptide fusion partners. Examples include the Staphylococcus protein A and the outer membrane protease IgA of Neisseria (Hansson et al. (1992) J. Bacteriol. 174, 4239-4245 and Klauser et al. (1990) EMBO J. 9, 1991-1999).

In the filamentous phage systems and the LamB system described above, the physical link between the peptide and its encoding DNA occurs by the containment of the DNA within a particle (cell or phage) that carries the peptide on its surface. Capturing the peptide captures the particle and the DNA within. An alternative scheme uses the DNA-binding protein LacI to form a link between peptide and DNA (Cull et al. (1992) PNAS USA 89:1865-1869). This system uses a plasmid containing the LacI gene with an oligonucleotide cloning site at its 3′-end. Under the controlled induction by arabinose, a LacI-peptide fusion protein is produced. This fusion retains the natural ability of LacI to bind to a short DNA sequence known as LacO operator (LacO). By installing two copies of LacO on the expression plasmid, the LacI-peptide fusion binds tightly to the plasmid that encoded it. Because the plasmids in each cell contain only a single oligonucleotide sequence and each cell expresses only a single peptide sequence, the peptides become specifically and stably associated with the DNA sequence that directed its synthesis. The cells of the library are gently lysed and the peptide-DNA complexes are exposed to a matrix of immobilized receptor to recover the complexes containing active peptides. The associated plasmid DNA is then reintroduced into cells for amplification and DNA sequencing to determine the identity of the peptide ligands. As a demonstration of the practical utility of the method, a large random library of dodecapeptides was made and selected on a monoclonal antibody raised against the opioid peptide dynorphin B. A cohort of peptides was recovered, all related by a consensus sequence corresponding to a six-residue portion of dynorphin B. (Cull et al. (1992) Proc. Natl. Acad. Sci. U.S.A. 89-1869)

This scheme, sometimes referred to as peptides-on-plasmids, differs in two important ways from the phage display methods. First, the peptides are attached to the C-terminus of the fusion protein, resulting in the display of the library members as peptides having free carboxy termini. Both of the filamentous phage coat proteins, pIII and pVIII, are anchored to the phage through their C-termini, and the guest peptides are placed into the outward-extending N-terminal domains. In some designs, the phage-displayed peptides are presented right at the amino terminus of the fusion protein. (Cwirla, et al. (1990) Proc. Natl. Acad. Sci. U.S.A. 87, 6378-6382) A second difference is the set of biological biases affecting the population of peptides actually present in the libraries. The LacI fusion molecules are confined to the cytoplasm of the host cells. The phage coat fusions are exposed briefly to the cytoplasm during translation but are rapidly secreted through the inner membrane into the periplasmic compartment, remaining anchored in the membrane by their C-terminal hydrophobic domains, with the N-termini, containing the peptides, protruding into the periplasm while awaiting assembly into phage particles. The peptides in the LacI and phage libraries may differ significantly as a result of their exposure to different proteolytic activities. The phage coat proteins require transport across the inner membrane and signal peptidase processing as a prelude to incorporation into phage. Certain peptides exert a deleterious effect on these processes and are underrepresented in the libraries (Gallop et al. (1994) J. Med. Chem. 37(9):1233-1251). These particular biases are not a factor in the LacI display system.

The number of small peptides available in recombinant random libraries is enormous. Libraries of 10⁷-10⁹ independent clones are routinely prepared. Libraries as large as 10¹¹ recombinants have been created, but this size approaches the practical limit for clone libraries. This limitation in library size occurs at the step of transforming the DNA containing randomized segments into the host bacterial cells. To circumvent this limitation, an in vitro system based on the display of nascent peptides in polysome complexes has recently been developed. This display library method has the potential of producing libraries 3-6 orders of magnitude larger than the currently available phage/phagemid or plasmid libraries. Furthermore, the construction of the libraries, expression of the peptides, and screening, is done in an entirely cell-free format.

In one application of this method (Gallop et al. (1994) J. Med. Chem. 37(9):1233-1251), a molecular DNA library encoding 10¹² decapeptides was constructed and the library expressed in an E. coli S30 in vitro coupled transcription/translation system. Conditions were chosen to stall the ribosomes on the mRNA, causing the accumulation of a substantial proportion of the RNA in polysomes and yielding complexes containing nascent peptides still linked to their encoding RNA. The polysomes are sufficiently robust to be affinity purified on immobilized receptors in much the same way as the more conventional recombinant peptide display libraries are screened. RNA from the bound complexes is recovered, converted to cDNA, and amplified by PCR to produce a template for the next round of synthesis and screening. The polysome display method can be coupled to the phage display system. Following several rounds of screening, cDNA from the enriched pool of polysomes was cloned into a phagemid vector. This vector serves as both a peptide expression vector, displaying peptides fused to the coat proteins, and as a DNA sequencing vector for peptide identification. By expressing the polysome-derived peptides on phage, one can either continue the affinity selection procedure in this format or assay the peptides on individual clones for binding activity in a phage ELISA, or for binding specificity in a completion phage ELISA (Barret, et al. (1992) Anal. Biochem 204,357-364). To identify the sequences of the active peptides one sequences the DNA produced by the phagemid host.

Assays for Activity

The high through-put assays described above can be followed (or substituted) by secondary screens, e.g., the following screens, in order to identify biological activities which will, e.g., allow one skilled in the art to differentiate agonists from antagonists. The type of a secondary screen used will depend on the desired activity that needs to be tested. Several such assays are described below. For example, an assay can be developed in which the ability to inhibit an interaction between a protein of interest (e.g., PKCβ) and a ligand (e.g., Rb) can be used to identify antagonists from a group of peptide fragments isolated though one of the primary screens described above.

Binding assays can be used to evaluate PKC, e.g., PKCβ, or Rb activity. E.g., PKCβ and Rb interact with each other and Rb and E2F interact with each other. Thus, the ability of one component to bind a binding partner is an assayable activity of PKC, e.g., PKCβ, or Rb activity. Thus, a binding assay, e.g., a binding assay described herein, can be used to evaluate: (a) the ability of a test agent to bind PKC, e.g., PKCβ, or Rb; (b) the ability of a test agent to inhibit binding of component to a binding partner, e.g., the ability of a test agent to inhibit or disrupt PKCβ/Rb or Rb/E2F interaction; (c) the ability of a test agent to stabilize or increase binding of a component to a binding partner, e.g., the ability of a test agent to stabilize or increase a PKCβ/Rb or Rb/E2F interaction.

As PKCβ and Rb can be purified, e.g., from mammals and/or have been cloned and produced recombinantly, they are readily available as reagents to be used in standard binding assays known in the art, which include, but are not limited to: affinity chromatography, size exclusion chromatography, gel filtration, fluid phase binding assay; ELISA (e.g., competition ELISA), immunoprecipitation. Such techniques are well known in the art.

PKC, e.g., PKCβ, and/or Rb activity can also be evaluated by measuring an enzymatic activity, e.g., by measuring PKC kinase activity. For example, PKC kinase activity can be assayed by evaluating the extent of ser/thr phosphorylation, e.g., in vitro, of a PKC substrate, e.g., Rb. Standard kinase assays can be used for this purpose.

Other assays, such as the cell growth and/or cell migration assay described herein, can also be used to test the activity of a test agent.

Administration

An agent that modulates PKC, e.g., PKCβ, or Rb, e.g., an agent described herein, can be administered to a subject by standard methods. For example, the agent can be administered by any of a number of different routes including intravenous, intradermal, subcutaneous, oral (e.g., inhalation), transdermal (topical), and transmucosal. In one embodiment, the modulating agent can be administered orally. In another embodiment, the agent is administered by injection, e.g., intramuscularly, or intravenously. In preferred embodiments, the agent is targeted, e.g., includes a targeting reagent, to a retinal tissue, an arthritic tissue, or a cancer tissue.

Any agent that modulates PKC, e.g., PKCβ, or Rb, e.g., an agent described herein, e.g., nucleic acid molecules, polypeptides, fragments or analogs, modulators, organic compounds and antibodies (also referred to herein as “active compounds”) can be incorporated into pharmaceutical compositions suitable for administration to a subject, e.g., a human. Such compositions typically include the nucleic acid molecule, polypeptide, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The nucleic acid molecules described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., PNAS 91:3054-3057, 1994). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

In a preferred embodiment, the pharmaceutical composition is administered directly into a retinal tissue, arthritic tissue, or tumor tissue of the subject.

Gene Therapy

The nucleic acids described herein, e.g., an antisense nucleic acid described herein, can be incorporated into gene constructs to be used as a part of a gene therapy protocol to deliver nucleic acids encoding either an agonistic or antagonistic form of PKC, e.g., PKCβ, or Rb. The invention features expression vectors for in vivo transfection and expression of a PKCβ, Rb or E2F described herein in particular cell types so as to reconstitute the function of, or alternatively, antagonize the function of the component in a cell in which that polypeptide is misexpressed. Expression constructs of such components may be administered in any biologically effective carrier, e.g. any formulation or composition capable of effectively delivering the component gene to cells, preferably adipose cells, in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO4 precipitation carried out in vivo.

A preferred approach for in vivo introduction of nucleic acid into a cell is by use of a viral vector containing nucleic acid, e.g. a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D. (1990) Blood 76:271). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include *Crip, *Cre, *2 and *Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol. 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of PKC, e.g., PKCβ, or Rb in the tissue of a subject. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al. (2001) J Invest Dermatol. 116(1):131-135; Cohen et al. (2000) Gene Ther 7(22):1896-905; or Tam et al. (2000) Gene Ther 7(21):1867-74.

In a representative embodiment, a gene encoding PKC, e.g., PKCβ, or Rb can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075).

In clinical settings, the gene delivery systems for the therapeutic gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

Cell Therapy

PKC, e.g., PKCβ, or Rb, can also be increased in a subject by introducing into a cell, e.g., an adipocyte, a nucleotide sequence that modulates the production of PKC, e.g., PKCβ, or Rb, e.g., a nucleotide sequence encoding PKC, e.g., PKCβ, or Rb, polypeptide or functional fragment or analog thereof, a promoter sequence, e.g., a promoter sequence from a PKC, e.g., PKCβ, or Rb gene or from another gene; an enhancer sequence, e.g., 5′ untranslated region (UTR), e.g., a 5′ UTR from a PKC, e.g., PKCβ, or Rb gene or from another gene, a 3′ UTR, e.g., a 3′ UTR from a PKC, e.g., PKCβ, or Rb gene or from another gene,; a polyadenylation site; an insulator sequence; or another sequence that modulates the expression of PKC, e.g., PKCβ, or Rb. The cell can then be introduced into the subject.

Primary and secondary cells to be genetically engineered can be obtained form a variety of tissues and include cell types which can be maintained propagated in culture. For example, primary and secondary cells include fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells (myoblasts) and precursors of these somatic cell types. Primary cells are preferably obtained from the individual to whom the genetically engineered primary or secondary cells are administered. However, primary cells may be obtained for a donor (other than the recipient). Preferred cells are endothelial cells, e.g., retinal endothelial cells.

The term “primary cell” includes cells present in a suspension of cells isolated from a vertebrate tissue source (prior to their being plated i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. Secondary cells are cell strains which consist of secondary cells which have been passaged one or more times.

Primary or secondary cells of vertebrate, particularly mammalian, origin can be transfected with an exogenous nucleic acid sequence which includes a nucleic acid sequence encoding a signal peptide, and/or a heterologous nucleic acid sequence, e.g., encoding PKC, e.g., PKCβ, or Rb, or an agonist or antagonist thereof, and produce the encoded product stably and reproducibly in vitro and in vivo, over extended periods of time. A heterologous amino acid can also be a regulatory sequence, e.g., a promoter, which causes expression, e.g., inducible expression or upregulation, of an endogenous sequence. An exogenous nucleic acid sequence can be introduced into a primary or secondary cell by homologous recombination as described, for example, in U.S. Pat. No.: 5,641,670, the contents of which are incorporated herein by reference. The transfected primary or secondary cells may also include DNA encoding a selectable marker which confers a selectable phenotype upon them, facilitating their identification and isolation.

Vertebrate tissue can be obtained by standard methods such a punch biopsy or other surgical methods of obtaining a tissue source of the primary cell type of interest. For example, punch biopsy is used to obtain skin as a source of fibroblasts or keratinocytes. A mixture of primary cells is obtained from the tissue, using known methods, such as enzymatic digestion or explanting. If enzymatic digestion is used, enzymes such as collagenase, hyaluronidase, dispase, pronase, trypsin, elastase and chymotrypsin can be used.

The resulting primary cell mixture can be transfected directly or it can be cultured first, removed from the culture plate and resuspended before transfection is carried out. Primary cells or secondary cells are combined with exogenous nucleic acid sequence to, e.g., stably integrate into their genomes, and treated in order to accomplish transfection. As used herein, the term “transfection” includes a variety of techniques for introducing an exogenous nucleic acid into a cell including calcium phosphate or calcium chloride precipitation, microinjection, DEAE-dextrin-mediated transfection, lipofection or electrophoration, all of which are routine in the art.

Transfected primary or secondary cells undergo sufficient number doubling to produce either a clonal cell strain or a heterogeneous cell strain of sufficient size to provide the therapeutic protein to an individual in effective amounts. The number of required cells in a transfected clonal heterogeneous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient.

The transfected cells, e.g., cells produced as described herein, can be introduced into an individual to whom the product is to be delivered. Various routes of administration and various sites (e.g., renal sub capsular, subcutaneous, central nervous system (including intrathecal), intravascular, intrahepatic, intrasplanchnic, intraperitoneal (including intraomental), intramuscularly implantation) can be used. One implanted in individual, the transfected cells produce the product encoded by the heterologous DNA or are affected by the heterologous DNA itself. For example, an individual who suffers from a retinopathy is a candidate for implantation of cells producing an antagonist of PKCβ, Rb or E2F described herein.

An immunosuppressive agent e.g., drug, or antibody, can be administered to a subject at a dosage sufficient to achieve the desired therapeutic effect (e.g., inhibition of rejection of the cells). Dosage ranges for immunosuppressive drugs are known in the art. See, e.g., Freed et al. (1992) N. Engl. J. Med. 327:1549; Spencer et al. (1992) N. Engl. J. Med. 327:1541′ Widner et al. (1992) n. Engl. J. Med. 327:1556). Dosage values may vary according to factors such as the disease state, age, sex, and weight of the individual.

Diagnostic Assays

The diagnostic assays described herein involve evaluating PKCβ, Rb or E2F levels, expression or activity in the subject. Various art-recognized methods are available for evaluating the activity of PKCβ, Rb or E2F. Techniques for detection of each of PKCβ, Rb or E2F are known in the art and include, inter alia: Western blot analysis, agar gel diffusion, radial immunodiffusion (RID), enzyme linked immunosorbent assays (ELISA), enzyme immunoassays (EIA), radioimmunoassays (RIA). Typically, the level in the subject is compared to the level and/or activity in a control, e.g., the level and/or activity in a tissue from a normal subject. Techniques for evaluating binding activity include fluid phase binding assays, affinity chromatography (e.g., Rb-sepharose chromatography), size exclusion or gel filtration, ELISA, immunoprecipitation.

Another method of evaluating PKCβ, Rb or E2F in a subject is to determine the presence or absence of a lesion in or the misexpression of a gene which encodes PKCβ, Rb or E2F. The method includes one or more of the following:

detecting, in a tissue of the subject, the presence or absence of a mutation which affects the expression of a gene encoding PKCβ, Rb or E2F, or detecting the presence or absence of a mutation in a region which controls the expression of the gene, e.g., a mutation in the 5′ control region;

detecting, in a tissue of the subject, the presence or absence of a mutation which alters the structure of a gene encoding PKCβ, Rb or E2F;

detecting, in a tissue of the subject, the misexpression of a gene encoding PKCβ, Rb or E2F, at the mRNA level, e.g., detecting a non-wild type level of a mRNA;

detecting, in a tissue of the subject, the misexpression of the gene, at the protein level, e.g., detecting a non-wild type level of a PKCβ, Rb or E2F polypeptide.

In preferred embodiments the method includes: ascertaining the existence of at least one of: a deletion of one or more nucleotides from a gene encoding PKCβ, Rb or E2F; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from a gene encoding PKCβ, Rb or E2F, or naturally occurring mutants thereof or 5′ or 3′ flanking sequences naturally associated with the gene; (ii) exposing the probe/primer to nucleic acid of a tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of a PKCβ, Rb or E2F gene; the presence of a non-wild type splicing pattern of a messenger RNA transcript of the gene; or a non-wild type level of a gene encoding PKCβ, Rb or E2F.

Methods of the invention can be used prenatally or to determine if a subject's offspring will be at risk for a disorder.

In preferred embodiments the method includes determining the structure of a gene encoding PKCβ, Rb or E2F, an abnormal structure being indicative of risk for the disorder.

In preferred embodiments the method includes contacting a sample from the subject with an antibody to PKCβ, Rb or E2F, or a nucleic acid which hybridizes specifically with the gene.

Expression Monitoring and Profiling.

The presence, level, or absence of PKCβ, Rb or E2F (protein or nucleic acid) in a biological sample can be evaluated by obtaining a biological sample from a test subject and contacting the biological sample with a compound or an agent capable of detecting the protein or nucleic acid (e.g., mRNA, genomic DNA) that encodes PKCβ, Rb or E2F such that the presence of the protein or nucleic acid is detected in the biological sample. The term “biological sample” includes tissues, cells and biological fluids isolated from a subject, as well as tissues, cells and fluids present within a subject, e.g., synovial fluid. Preferred biological samples are serum or synovial fluid. The level of expression of PKCβ, Rb or E2F can be measured in a number of ways, including, but not limited to: measuring the mRNA encoded by the PKCβ, Rb or E2F gene; measuring the amount of protein encoded by a PKCβ, Rb or E2F gene; or measuring the activity of the protein encoded by the gene.

The level of mRNA corresponding to a PKCβ, Rb or E2F gene in a cell can be determined both by in situ and by in vitro formats.

Isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length nucleic acid, or a portion thereof, such as an oligonucleotide of at least 7, 15, 30, 50, 100, 250 or 500 nucleotides in length and sufficient to specifically hybridize under stringent conditions to mRNA or genomic DNA of a PKCβ, Rb or E2F gene. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the PKCβ, Rb or E2F gene.

The level of mRNA in a sample that is encoded by a gene can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the gene being analyzed.

In another embodiment, the methods further contacting a control sample with a compound or agent capable of detecting mRNA, or genomic DNA of a PKCβ, Rb or E2F gene, and comparing the presence of the mRNA or genomic DNA in the control sample with the presence of mRNA or genomic DNA of PKCβ, Rb or E2F in the test sample. In still another embodiment, serial analysis of gene expression, as described in U.S. Pat. No. 5,695,937, is used to detect transcript levels of PKCβ, Rb or E2F.

A variety of methods can be used to determine the level of protein encoded by a PKCβ, Rb or E2F gene. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect a PKCβ, Rb or E2F in a biological sample in vitro as well as in vivo. In vitro techniques for detection of component of PKCβ, Rb or E2F include enzyme linked immunosorbent assays (ELISAs), immunoprecipitations, immunofluorescence, enzyme immunoassay (EIA), radioimmunoassay (RIA), and Western blot analysis. In vivo techniques for detection of PKCβ, Rb or E2F include introducing into a subject a labeled anti-PKCβ, Rb or E2F antibody. For example, the antibody can be labeled with a radioactive marker whose presence and location in a subject can be detected by standard imaging techniques. In another embodiment, the sample is labeled, e.g., biotinylated and then contacted to the antibody, e.g., an antibody positioned on an antibody array. The sample can be detected, e.g., with avidin coupled to a fluorescent label.

In another embodiment, the methods further include contacting the control sample with a compound or agent capable of detecting PKCβ, Rb or E2F, and comparing the presence of the component protein in the control sample with the presence of PKCβ, Rb or E2F protein in the test sample.

The invention also includes kits for detecting the presence of PKCβ, Rb or E2F in a biological sample. For example, the kit can include a compound or agent capable of detecting protein (e.g., an antibody) or mRNA (e.g., a nucleic acid probe) of PKCβ, Rb or E2F in a biological sample; and a standard. The compound or agent can be packaged in a suitable container. The kit can further comprise instructions for using the kit to evaluate a subject, e.g., for risk or predisposition to a retinopathy, e.g., a retinopathy described herein.

The diagnostic methods described herein can identify subjects having, or at risk of developing, a retinopathy, e.g., a retinopathy described herein.

The prognostic assays described herein can be used to determine whether a subject can be administered an agent (e.g., an agent that inhibits VEGF-mediated angiogenesis, e.g., an agent described herein) to treat a retinopathy, e.g., a retinopathy described herein.

This invention is further illustrated by the following examples that should not be construed as limiting. The contents of all references, patents and published patent applications cited throughout this application are incorporated herein by reference.

### EXAMPLES

### Example 1

### Mice

Linearized PEP8-PKC_{—}2 DNA was used to derive transgenic mice as described (9-11). PKCβ2 cDNA probe was used to examine the incorporation of the transgene. Transgene expression was confirmed by Northern blot and immunoblot on heart, aorta, and retina. Founders were bred with C57BL/6 mice and F1 mice were used for experiments. PKCβ null mice are known in the art (12).

### Example 2

### Ischemia-Induced Proliferative Retinopathy

The study adhered to the Association for Research in Vision and Ophthalmology statement for the Use of Animals in Ophthalmic and Vision Research. Postnatal day 7 (P7) mice and their nursing mothers were exposed to 75±2% oxygen for 5 days to induce and then returned to room air inducing retinal vaso-obliteration. Maximum neovascularization was observed at P17. Flat-mounted, fluorescein-conjugated dextran-perfused retinas were examined to assess the retinal vasculature (see refs. 13 and 14).

### Example 3

### Quantitation of Neovascularization

Mice at P17 were killed and eyes enucleated, fixed, and embedded in paraffin. Fifty serial sections (6 μm) starting at the optic nerve head were placed on microscope slides. After staining with periodic acid/Schiff re-agent and hematoxylin, 10 intact sections of equal length, each 30 μm apart, were evaluated for a span of 300 μm. All retinal vascular cell nuclei anterior to the internal limiting membrane were counted in each section by a fully masked protocol. The mean of all 10 counted sections yielded average neovascular cell nuclei per 6-μm section per eye (see refs. 13 and 14). Northern and Western Blot Analysis. Total RNA or protein from 8-10 retinas at P14 was assessed by Northern (15) or Western (16) blot analysis as described.

### Example 4

### Cell Culture

Primary cultures of bovine retinal endothelial cells (BREC) were isolated and cultured as described (17). Only cells from passages 2-7 were used for the experiments.

### Example 5

### Recombinant Adenoviruses

cDNA of dominant-negative PKCs was constructed as described (18-21). The replication-deficient recombinant adenoviruses were constructed by homologous recombination between the parental virus genome and shuttle vector as described (22). The dominant-negatives of PKCβ2 isoform were constructed by converting threonine-500 to valine which caused the PKC isoform to become kinase-inactive. The adenoviruses were applied at a concentration of 1×10 8 plaque-forming units/ml, and adenoviruses with the same parental genome carrying enhanced green fluorescent protein (EGFP) gene were used as controls. Infection efficiency was monitored by fluorescence which showed expression in >80% of cells. Expression of each recombinant protein was confirmed by Western blot analysis of each PKC isoform. Both the wild types and their respective dominant-negative isoforms exhibited increases 8- to 10-fold above the endogenous isoforms by protein levels.

### Example 6

### Cell Growth Assay

BREC were plated onto 12-well culture plates and incubated overnight in DMEM containing 10% calf serum, after which the cells were infected with adenovirus (23). After incubation for 4 days at 37° C. with or without VEGF (0.6 nM), the cells were lysed in 0.1% SDS, and the DNA content was measured by means of Hoechst-33258 dye and a fluorometer (model TKO-100, Hoefer). E2 promoter binding factor (E2F) decoys were used as described (24).

### Example 7

### Cell Migration Assay

A modified Boyden chamber migration assay was performed by using BREC (25). The top and bottom surface of the chamber membrane was coated with collagen I. Serum-starved BREC overexpressing each PKC isoform were induced to migrate toward VEGF (0.6 nM) placed in the bottom chamber and harvested after 4 h. Cells that migrated to the bottom of the chamber were enumerated by counting SYTOX green (Molecular Probes) nucleic acid-stained cells.

### Example 8

### In Vitro Phosphorylation of Retinoblastoma (Rb) Protein.

In vitro phosphorylation of Rb protein by recombinant PKC (Upstate Biotechnology, Lake Placid, N.Y.) was performed as described (26) with recombinant Rb protein (QED Bioscience, San Diego) as a substrate.

### Example 9

### Luciferase Assay

Luciferase reporter constructs (Mercury Cell Cycle Profiling System, CLONTECH) were introduced into cells with LipofectAMINE reagent (Life Technologies, Rockville, Md.) as instructed by the manufacturer. Luciferase activity was measured by using the Dual-Luciferase Reporter Assay system (Promega).

### REFERENCES

1. Wu et al (2000) J. Biol. Chem. 275, 5096-5103.

2. Xia et al. (1996) J. Clin. Invest. 98, 2018-2026.

3. Takahashi et al. (1999) Oncogene 18, 2221-2230.

4. Kim et al. (1993) Nature (London) 362, 841-844.

5. Plate et al.(1992) Nature (London) 359, 845-848.

6. Firestein et al. (1999) J. Clin. Invest. 103, 3-4.

7. Aiello et al. (1997) Diabetes 46, 1473-1480.

8. Aiello et al. (1994) N. Engl. J. Med. 331, 1480-1487.

9. Harats et al. (1995) J. Clin. Invest. 95, 1335-1344.

10. Ohashi et al. (1998) J. Clin. Invest. 102, 2061-2071.

11. Wakasaki et al. (1997) Proc. Natl. Acad. Sci. USA 94, 9320-9325.

12. Leitges et al. (1996) Science 273, 788-791.

13. Aiello et al. (1995) Proc. Natl. Acad. Sci. USA 92, 10457-10461.

14. Smith et al. (1994) Invest. Ophthalmol. Visual Sci. 35, 101-111.

15. Takagi et al. (1996) Diabetes 45, 1016-1023.

16. Jiang et al. (1999) J. Clin. Invest. 104, 447-457.

17. King et al. (1985) J. Clin. Invest. 75, 1028-1036.

18. Uberall et al. (1997) J. Biol. Chem. 272, 4072-4078.

19. Orr et al. (1994) J. Biol. Chem. 269, 27715-27718.

20. Li et al. (1995) J. Biol. Chem. 270, 8311-8318.

21. Uberall et al. (1999) J. Cell Biol. 144, 413-425.

22. He et al. (1998) Proc. Natl. Acad. Sci. USA 95, 2509-2514.

23. Thieme et al. (1995) Diabetes 44, 98-103.

24. Morishita et al. (1995) Proc. Natl. Acad. Sci. USA 92, 5855-5859.

25. Sakamoto et al. (1995) Invest. Ophthalmol. Visual Sci. 36, 1076-1083.

26. Ishii et al. (1996) Science 272, 728-731.

27. Kaelin (1999) BioEssays 21, 950-958.

28. Fields et al. (1988) J. Biol. Chem. 263, 8253-8260.

29. Connell-Crowley et al. (1997) Mol. Biol Cell 8, 287-301.

30. Zarkowska & Mittnacht (1997) J. Biol. Chem. 272, 12738-12746.

31. Brown et al. (1999) Mol. Cell. Biol. 19, 3246-3256.

32. Knudsen & Wang (1997) Mol. Cell. Biol. 17, 5771-5783.

We claim:

1. A method of modulating angiogenesis in a cell, tissue, or subject comprising: modulating a PKCβ activity in a cell, tissue, or subject.

2. The method of claim 1, wherein the method comprises administering to the cell, tissue or subject an agent that inhibits PKCβ, to thereby decrease angiogenesis.

3. The method of claim 2, wherein the agent is LY-333531.

4. The method of claim 2, wherein the agent is a dominant negative PKCβ polypeptide.

5. The method of claim 2, wherein the agent is nucleic acid that decreases or silences expression of a PKCβ gene.

6. The method of claim 1, wherein the cell or tissue is a retinal cell or tissue.

7. The method of claim 6, wherein the retinal tissue is ischemic retinal tissue.

8. The method of claim 1, wherein the cell or tissue is a tumor cell or tissue.

9. The method of claim 1, wherein the subject is a human.

10. The method of claim 1, wherein the subject is an experimental animal.

11. The method of claim 1, wherein the method comprises administering to the cell, tissue or subject an agent that increases a PKCβ activity, to thereby increase angiogenesis.

12. The method of claim 11, wherein the agent is a PKCβ polypeptide or functional fragment thereof.

13. The method of claim 11, wherein the agent is a nucleic acid encoding a PKCβ polypeptide or functional fragment thereof.

14. The method of claim 11, wherein the subject is a human.

15. The method of claim 11, wherein the subject is an experimental animal.

16. A method of treating an angiogenesis-related disorder in a subject, the method comprising:
(a) identifying a subject in need of prevention or treatment for an angiogenesis-related disorder; and
(b) administering to the subject an agent that decreases PKCβ activity in a cell or tissue of the subject.

17. The method of claim 16, wherein the disorder is retinopathy.

18. The method of claim 16, wherein the disorder is rheumatoid arthritis.

19. The method of claim 16, wherein the disorder is a tumor.

20. The method of claim 16, wherein the agent is LY333531.

21. The method of claim 16, wherein the agent is a dominant negative PKCβ polypeptide.

22. The method of claim 16, wherein the agent is nucleic acid that decreases or silences expression of a PKCβ gene.

23. The method of claim 16, wherein the PKCβ is PKCβ2.
